(19) United States
(12) Patent Application Publication
(10) Pub. No.: US 2026/0048233 A1
(43) Pub. Date: Feb. 19, 2026

(54) CATHETER SHAFT AND METHODS OF MAKING A CATHETER SHAFT

(71) Applicant: Boston Scientific Scimed, Inc., Maple Grove, MN (US)

(72) Inventors: Emily Rose Luecke, Osseo, MN (US); Jason John Matteson, Beldenville, WI (US); Cory P. Wright, Woodbury, MN (US)

(21) Appl. No.: 19/300,993

(22) Filed: Aug. 15, 2025

Related U.S. Application Data
(60) Provisional application No. 63/684,067, filed on Aug. 16, 2024.

(51) Int. Cl. A61M 25/00; A61M 25/01

(52) U.S. Cl. CPC A61M 25/0053; A61M 25/0012; A61M 25/0136; A61M 25/0138

(57) ABSTRACT

A medical catheter with improved structural integration and performance is disclosed. The catheter features a flexible shaft with a reinforcing braid and a polymeric jacket. Structural elements, such as a reinforcing assembly and an articulation structure, are positioned within the shaft and mechanically connected to the polymeric jacket. The connection is formed by creating windows in the polymeric jacket to expose the underlying reinforcing braid and disposing an attachment material through the windows and the braid into circumferential grooves of the structural elements. The attachment material fills the grooves, interlocking the structural elements with the braid and creating a strong, seamless bond with the polymeric jacket. This approach enhances force transmission, steering control, and overall catheter performance. The mechanical connection can be customized by varying the size, shape, and location of the windows and grooves, making the design adaptable to various catheter configurations and applications.

### CROSS REFERENCE TO RELATED APPLICATIONS

This application claims priority to U.S. Provisional Patent Application No. 63/684,067 entitled “BOWDEN RING SHAFT ATTACHMENT 360DEGREE GROOVE,” filed Aug. 16, 2024, which is hereby incorporated by reference in its entirety.

### TECHNICAL FIELD

The present invention relates to medical catheters and methods for manufacturing medical catheters. More specifically, to medical catheters with enhanced structural integration and performance through mechanical connections of structural elements such as a reinforcing assembly or an articulation structure.

### BACKGROUND

Medical catheters are commonly used in minimally invasive medical procedures to navigate through complex anatomical pathways and access target sites within the body. These catheters typically include a flexible elongated shaft with a steerable distal portion that can be deflected or curved by the operator to facilitate navigation and positioning. The steering mechanism often involves the use of pull wires or other tensioning elements that extend through the catheter shaft and connect to the distal portion, allowing the operator to control the deflection of the catheter tip.

To achieve optimal performance and maneuverability, catheters rely on the integration of various structural elements within the catheter shaft, such as Bowden assemblies for steering wire guidance or articulation structures for controlled deflection. However, securely anchoring these components within the catheter shaft can be challenging. Conventional methods of integrating structural elements often involve bonding them to the inner surface of the catheter shaft using adhesives or thermal techniques. These approaches may not provide sufficient mechanical stability or durability, especially in regions of the catheter subjected to high stress during steering and navigation. Additionally, the use of adhesives or thermal bonding can create variability in the outer diameter of the catheter shaft and localized stiffness or weakness, disrupting the desired gradual transition of mechanical properties along its length.

The present invention addresses these needs by utilizing exposed portions of the catheter shaft's reinforcing braid as anchoring points for structural elements. By selectively exposing the underlying reinforcing braid through a removal process and mechanically connecting the structural elements through the braid, it allows for structural integration without compromising the catheter's flexibility or introducing abrupt changes in stiffness. This approach enhances the structural continuity between the catheter shaft and structural elements such as the reinforcing assembly or articulation structure, resulting in improved steering responsiveness and overall catheter performance.

### SUMMARY

In Example 1, a medical catheter comprising a handle configured for manipulation by a user, and a flexible shaft comprising a reinforcing braid and a polymeric jacket disposed over the reinforcing braid, the flexible shaft having a proximal portion, and a distal portion, wherein a structural element is positioned within the shaft, wherein the structural element is mechanically connected to the polymeric jacket via a mechanical connection establishing a secure bond between the polymeric jacket and the structural element, and wherein the mechanical connection is formed by selectively removing a portion of the polymeric jacket to create a window exposing a portion of the reinforcing braid and disposing an attachment material through the window and the exposed portion of the reinforcing.

In Example 2, the medical catheter of Example 1, wherein the structural element comprises a circumferential groove configured to receive the attachment material.

In Example 3, the medical catheter of Example 2, wherein the circumferential groove is formed on an outer surface of the structural element.

In Example 4, the medical catheter of Example 2, wherein the circumferential groove is positioned in close proximity to the window of the exposed reinforcing braid.

In Example 5, the medical catheter of Example 4, wherein the attachment material is disposed through the window of the exposed portion of the reinforcing braid into the groove of the structural element to establish the mechanical connection.

In Example 6, the medical catheter of Example 5, wherein the attachment material is an adhesive, a polymeric material, or other similar bonding materials.

In Example 7, the medical catheter of Example 1, wherein the selective removal of the polymeric jacket is controlled to create a desired size and shape of the window of the exposed reinforcing braid.

In Example 8, the medical catheter of Example 1, wherein the structural element is a reinforcing assembly, wherein the reinforcing assembly is positioned within the proximal portion of the shaft, wherein the reinforcing assembly comprises a pair of diametrically opposed reinforcing sleeves extending through the proximal portion of the shaft, wherein the reinforcing sleeves establish passageways configured for one or more steering elements extending from the proximal region to the distal region of the shaft, and an anchor ring attached to a distal end of each of the reinforcing sleeves, the anchor ring comprising a ring having a groove defined around its circumference.

In Example 9, the medical catheter of Example 8, further comprising a pair of diametrically opposed steering elements each extending through a respective one of the reinforcing sleeves, wherein the reinforcing sleeves each provide radial and lateral support for the steering elements extending therethrough.

In Example 10, the medical catheter of Example 8, wherein the anchor ring includes a pair of diametrically opposed passageways aligned with the passageways of the reinforcing sleeves, and when each of the steering elements extends through a respective one of the passageways in the anchor ring.

In Example 11, the medical catheter of Example 10, wherein the anchor ring of the reinforcing assembly is positioned in close proximity to the window of the exposed reinforcing braid.

In Example 12, the medical catheter of Example 11, wherein the attachment material is disposed through the window of the exposed reinforcing braid into the groove of the anchor ring to establish the mechanical connection.

In Example 13, the medical catheter of Example 1, wherein the structural element is an articulation structure positioned within the distal portion of the shaft, the articulation structure having a proximal end, wherein the articulation structure comprises a plurality of longitudinally-arranged articulation elements, one or more steering wire lumens, wherein the steering wire lumens extend through the articulation structure, one or more reinforcing members extending through the articulation elements, and a circumferential groove formed on the proximal end of the articulation structure.

In Example 14, the medical catheter of Example 13, wherein the articulation elements comprise a plurality of tubular segments, a plurality of connecting segments, a plurality of articulation links, a plurality of joints, a plurality of reinforcing members, or combinations thereof.

In Example 15, the medical catheter of Example 14, wherein the attachment material is disposed through the window of the exposed reinforcing braid into the groove of the articulation structure to establish the mechanical connection.

In Example 16, a medical catheter comprising a handle configured for manipulation by a user, and a flexible shaft comprising a reinforcing braid and a polymeric jacket disposed over the reinforcing braid, the flexible shaft having a proximal portion and a distal portion, the shaft further comprising a reinforcing assembly extending within the proximal portion of the shaft, wherein the reinforcing assembly comprises an anchor ring and a pair of diametrically opposed reinforcing sleeves attached to and extending proximally from the anchor ring within the proximal portion of the shaft, and wherein the anchor ring includes a circumferential groove, wherein the anchor ring is mechanically connected to the polymeric jacket and the reinforcing braid via a first mechanical connection establishing a secure bond between the polymeric jacket, the reinforcing braid and the structural element, and wherein the first mechanical connection is formed by disposing an attachment material into the circumferential groove in the anchor ring through a first window formed in the polymeric jacket.

In Example 17, the medical catheter of Example 16, wherein the attachment material is an adhesive, a polymeric material, or other similar bonding materials.

In Example 18, the medical catheter of Example 17, further comprising a pair of diametrically opposed steering elements each extending through a respective one of the reinforcing sleeves, wherein the reinforcing sleeves each provide radial and lateral support for the steering elements extending therethrough.

In Example 19, the medical catheter of Example 18, wherein the anchor ring includes a pair of diametrically opposed passageways aligned with the passageways of the reinforcing sleeves, and when each of the steering elements extends through a respective one of the passageways in the anchor ring.

In Example 20, the medical catheter of Example 19, wherein the shaft further comprises an articulation structure positioned within the distal portion of the shaft, the articulation structure having a proximal end positioned distally adjacent to the anchor ring.

In Example 21, the medical catheter of Example 20, wherein the proximal end of the articulation structure includes a circumferential groove and is mechanically connected to the polymeric jacket and the reinforcing braid via a second mechanical connection formed by disposing an attachment material into the circumferential groove in the proximal end of the articulation structure through a second window formed in the polymeric jacket.

In Example 22, the medical catheter of Example 21, wherein the articulation structure includes a plurality of longitudinally-arranged articulation elements, and a pair of diametrically opposed steering wire lumens, each of the steering wire lumens being aligned with a respective one of the reinforcing sleeves, and wherein each of the steering elements extends through a respective one of the steering wire lumens.

In Example 23, a medical catheter comprising a handle configured for manipulation by a user, and a flexible shaft comprising a reinforcing braid and a polymeric jacket disposed over the reinforcing braid, the flexible shaft having a proximal portion and a distal portion, the shaft further comprising a reinforcing assembly extending within the proximal portion of the shaft, wherein the reinforcing assembly comprises an anchor ring and a pair of diametrically opposed reinforcing sleeves attached to and extending proximally from the anchor ring within the proximal portion of the shaft, and wherein the anchor ring includes a circumferential groove, wherein an adhesive attachment material is disposed within the circumferential groove in the anchor ring and mechanically connects the anchor ring to the reinforcing braid and the polymeric jacket.

In Example 24, the medical catheter of Example 23, further comprising a pair of diametrically opposed steering elements each extending through a respective one of the reinforcing sleeves, wherein the reinforcing sleeves each provide radial and lateral support for the steering elements extending therethrough.

In Example 25, the medical catheter of Example 24, wherein the anchor ring includes a pair of diametrically opposed passageways aligned with the passageways of the reinforcing sleeves, and when each of the steering elements extends through a respective one of the passageways in the anchor ring.

In Example 26, the medical catheter of Example 25, wherein the reinforcing sleeves comprise helical coils.

In Example 27, the medical catheter of Example 25, wherein the shaft further comprises an articulation structure positioned within the distal portion of the shaft, the articulation structure having a proximal end positioned distally adjacent to the anchor ring.

In Example 28, the medical catheter of Example 27, wherein the proximal end of the articulation structure includes a circumferential groove and is mechanically connected to the polymeric jacket and the reinforcing braid via an adhesive attachment material disposed within the circumferential groove in the proximal end of the articulation structure.

In Example 29, the medical catheter of Example 28, wherein the articulation structure includes a plurality of longitudinally-arranged articulation elements, and a pair of diametrically opposed steering wire lumens, each of the steering wire lumens being aligned with a respective one of the reinforcing sleeves, and wherein each of the steering elements extends through a respective one of the steering wire lumens.

In Example 30. The medical catheter of Example 29, wherein the articulation structure further includes a pair of diametrically opposed reinforcing members extending along a length of the articulation structure, wherein each of the reinforcing members is located 90 degrees from the steering wire lumens of the articulation structure.

In Example 31, a method of producing a medical catheter comprising providing a flexible shaft comprising a reinforcing braid and a polymeric jacket disposed over the reinforcing braid, the flexible shaft having a proximal portion, and a distal portion, removing a portion of the polymeric jacket to create a window of the exposed reinforcing braid, positioning a structural element within the shaft adjacent to the window, connecting the structural element to the polymeric jacket by disposing an attachment material through the window of the exposed reinforcing braid to establish a secure bond between the structural element, the reinforcing braid and the polymeric jacket, and securing a handle to the proximal portion of the catheter shaft, the handle configured for manipulation by a user.

In Example 32, the method of Example 31, wherein the structural element comprises a reinforcing assembly comprising an anchor ring and a pair of diametrically opposed reinforcing sleeves attached to and extending proximally from the anchor ring within the proximal portion of the shaft, and wherein positioning the structural element includes positioning the anchor ring adjacent to the window, and connecting the structural element to the polymeric jacket comprises disposing an adhesive material through the window to establish a secure bond between the anchor ring, the reinforcing braid and the polymeric jacket.

In Example 33, the method of Example 32, wherein the anchor ring includes a circumferential groove, and wherein connecting the structural element to the polymeric jacket includes disposing the adhesive material through the window into the circumferential groove.

In Example 34, the method of Example 33, wherein the reinforcing sleeves comprise helical coils and are each configured to provide radial and lateral support for a steering element extending therethrough.

In Example 35, the method of Example 31, wherein the structural element is an articulation structure having a proximal end having a circumferential groove formed thereon, a plurality of longitudinally-arranged articulation elements, and one or more steering wire lumens extending through the articulation structure, and wherein positioning the structural element includes positioning the proximal end of the articulation structure adjacent to the window, and connecting the structural element to the polymeric jacket comprises disposing an adhesive material through the window and into the circumferential groove to establish a secure bond between the proximal end of the articulation structure, the reinforcing braid and the polymeric jacket.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an illustration of an example medical catheter, consistent with various aspects of the present disclosure.

FIG. 2A is an illustration of the medical catheter, shown in FIG. 1, as deflected in a first direction, consistent with various aspects of the present disclosure.

FIG. 2B is an illustration of the medical catheter, shown in FIG. 1, as deflected in a second direction, consistent with various aspects of the present disclosure.

FIG. 3 is a detailed illustration of a portion of a side-view illustration of the medical catheter, consistent with various aspects of the present disclosure.

FIG. 4A is an illustration of a reinforcing assembly enclosed in the medical catheter with a grooved anchor ring attachment, consistent with various aspects of the present disclosure.

FIG. 4B is an illustration of an articulation structure enclosed in the medical catheter with a circumferential groove formed on its proximal end, consistent with various aspects of the present disclosure.

FIG. 5 is a top-view illustration of a reinforcing assembly of the medical catheter, consistent with various aspects of the present disclosure.

FIG. 6 is an illustration of an articulation structure with a circumferential groove on its proximal end and reinforcing assembly with a grooved anchor ring enclosed in the medical catheter, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

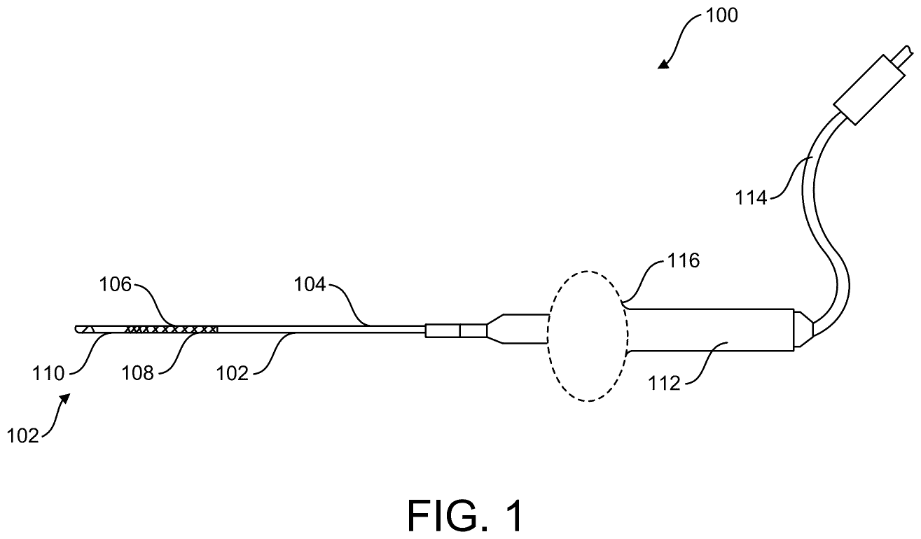

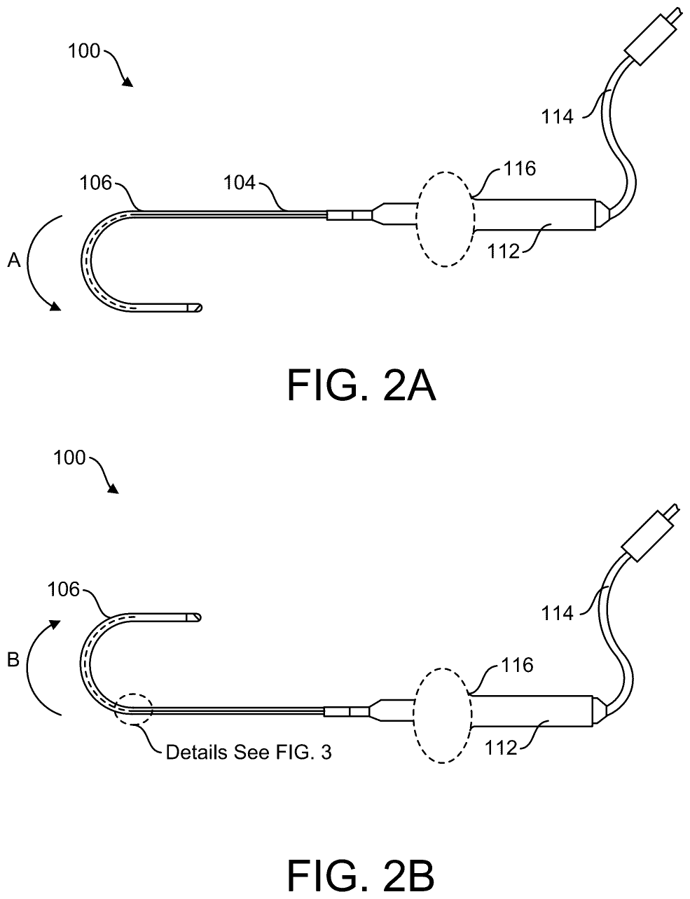

FIG. 1, FIG. 2A and FIG. 2B are illustrations of an example medical catheter 100, consistent with various aspects of the present disclosure. As shown in FIG. 1, the medical catheter 100 may be a steerable medical catheter 100. In certain instances, the medical catheter 100 is steerable in one direction (e.g., direction A as shown in FIG. 2A) or in another direction (e.g., direction B as shown in FIG. 2B). The medical catheter 100 generally includes a tubular shaft 102 having a proximal portion 104 and a distal portion 106 that is sized and configured for placement and manipulation within in a target area of a patient. The distal portion 106 may be steerable. As shown, the distal portion 106 further includes a deflection region 108 and a distal end 110.

In certain instances, the tubular shaft 102 extends from a distal portion of a handle 112. An electrical cable or other suitable connector 114 extending from a proximal end of the handle 112 may be coupled to a source of energy or other equipment (not shown in FIG. 1) for transmitting one or more ablation signals. A steering actuator 116, such as a rotatable knob or plunger that may be arranged at the handle 112, may be manipulated by a physician to deflect or position the steerable distal portion 106 of the tubular shaft 102.

As shown in FIGS. 2A and 2B, the medical catheter 100 is of the deflectable or steerable type, such that during use, the deflection region 108 can be deflected or curved by a user to facilitate locating the distal end 110 at a desired target location within the heart. In embodiments, deflection of the deflection region 108 can be accomplished by manipulation of the steering actuator 118, which is operatively connected to steering elements (e.g., wires, tendons, ribbons, and the like) extending within and attached (directly or indirectly) to the shaft 102 at a location within the distal portion 106. The particular mode and structure for deflecting the deflection region 108 is not critical to the present disclosure, and so any technique, whether now known or later developed, can be employed within the scope of the present disclosure.

In use, deflecting or curving the distal portion 106 may impart a torsional force that could torque or twist the distal portion 106 away from or out of the target location. The deflection region 108 may torque out of plane from the plane in which the distal portion 106 was arranged prior to deflection due to the tension on the curvature of the medical catheter 100 within vasculature.

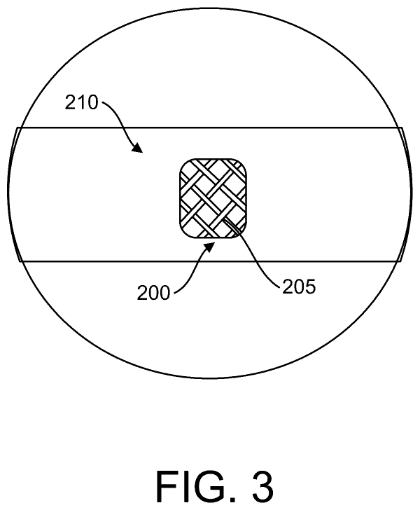

The catheter shaft 102 comprises a polymeric jacket 210 that surrounds and encapsulates the shaft 102, wherein the polymeric jacket 210 is composed of a polymeric material and includes an embedded reinforcing braid 205 formed of a plurality of interwoven wires that are woven, knitted, entwined or otherwise interlaced together. FIG. 3 is an enlarged detail view of the medical catheter 100, illustrating the removal of a portion of the polymeric jacket 210 to create a window 200 of the exposed reinforcing braid 205 on the catheter shaft 102. The window 200 of the exposed reinforcing braid 205 is created through a selective removal process wherein the removal process can be achieved through various processes, such as laser ablation, mechanical cutting, chemical etching, or thermal ablation. By carefully controlling the depth, shape, and size of the removal process, the window 200 of the exposed reinforcing braid 205 can be tailored to a desired dimension and can be formed at any location along the catheter shaft 102. In various embodiments, the size of the window 200 of the exposed reinforcing braid may be varied based on various factors relating to the structural characteristics and requirements of the catheter shaft 102, the manufacturing equipment and processes employed, and the like. In embodiments, the window 200 may be generally rectangular in shape, and have minimum dimensions of about 0.020 inches by 0.020 inches, or alternatively in embodiments, the window 200 may have a non-rectangular shape (e.g., circular, elliptical) with a minimum exposed braid area comparable to the aforementioned rectangular window 200.

In the illustrated embodiment, laser ablation is used to remove sections of the polymeric jacket 210 in a precise and controlled manner. The laser ablation parameters, such as wavelength, power, pulse duration, spot size, or other parameters are selected to effectively remove the polymeric material without causing damage to the underlying reinforcing braid 205. The laser is guided along the surface of the catheter shaft 102 to create the window 200 of the reinforcing braid 205. In some embodiments, the laser may create the window 200 of the reinforcing braid 205 with well-defined edges and a predetermined shape, such as a circle, oval, square, rectangle or other shapes.

The location and arrangement of the window 200 can be tailored to accommodate the specific structural elements that may be mechanically connected with the polymeric jacket 210. In the present disclosure, the window 200 of the exposed reinforcing braid 205 are strategically positioned to align with circumferential grooves formed on the outer surfaces of structural elements, such as a reinforcing assembly or an articulation structure. A mechanical connection 202 (connection show in later figures) may be established between the structural elements and the polymeric jacket 210 by introducing an attachment material such as an adhesive, polymeric material, or similar bonding materials through the window 200 of the exposed braids 205 into the circumferential grooves. The attachment material is flowed through the window 200 to fill the space of the circumferential groove, until the voids or gaps of the groove are filled. As the attachment material fills the groove, it creates a direct contact between the structural element and the polymeric jacket 210. This direct contact allows the attachment material to adhere to both the structural element and the braid 205, effectively locking the structural element in place and creating a strong bond between the structural element and the polymeric jacket. The attachment material, upon curing or solidifying, may become an integral part of the catheter shaft 102, seamlessly integrating the structural element into the shaft 102

In some embodiments, two windows 200 may be created, positioned on opposite sides of the shaft, 180-degree from the first window. The two windows allow for the attachment material to be introduced through the first window and flow into the circumferential groove. The attachment material fills the groove and continues to flow until the attachment material exits the second window, allowing the user to identify that the process of flowing the attachment material through the window is complete when the attachment material is observed to exit the second window. This ensures the user that the groove is entirely filled and no voids or gaps remain. As the attachment material fills the groove, it creates a direct contact between the structural element and the polymeric jacket 210. This direct contact allows the attachment material to adhere to both the structural element and the braid 205, effectively locking the structural element in place and creating a strong bond between the structural element and the polymeric jacket. The attachment material, upon curing or solidifying, may become an integral part of the catheter shaft 102, seamlessly integrating the structural element into the shaft 102.

The window 200 of the exposed reinforcing braid 205 may be created at any desired location along the length of the catheter shaft 102, enabling the positioning of structural elements and mechanically connecting the structural elements at optimal points for force transmission and steering control. Further, by controlling the removal process, the window 200 can be formed without significantly altering the outer diameter or surface profile of the catheter shaft 102, maintaining a smooth and consistent transition between the shaft and the structural elements.

In some embodiments, the removal process can be adapted to create the window 200 of various shapes and sizes, depending on the specific requirements of the catheter design and the structural elements being integrated. For example, elongated openings or slots may be formed to accommodate larger structural elements or to provide multiple points of mechanical connecting along the length of the structural element. Additionally, the number and arrangement of window 200 of the exposed reinforcing braid 205 can be varied to optimize the force distribution and steering performance of the catheter 100.

In some embodiments, the existing polymeric jacket 210 may be reflowed over the window 200 to seal and encapsulate the window 200. The reflowed polymeric material from the existing polymeric jacket 210 forms a smooth, continuous surface over the exposed reinforcing braid 204 and the integrated structural elements.

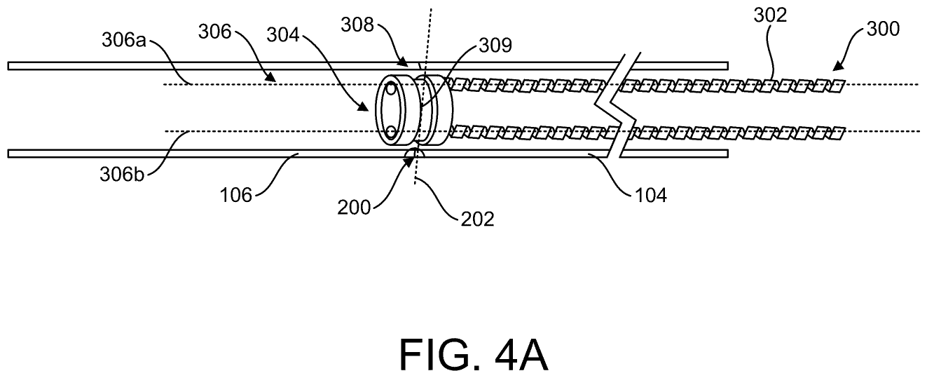

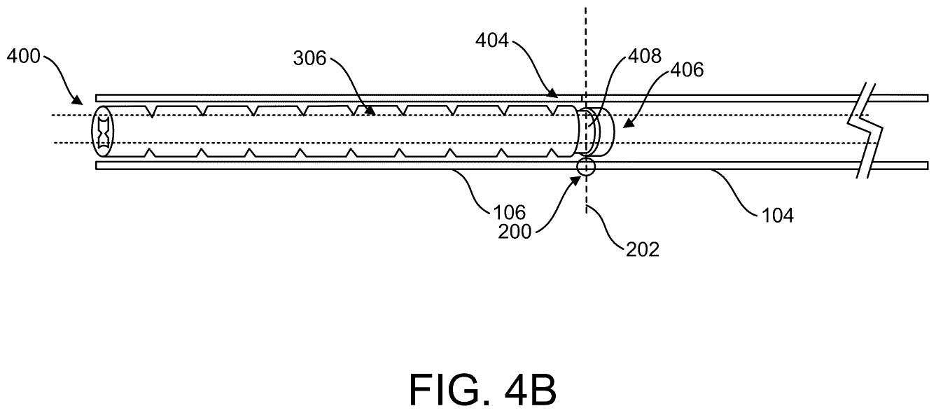

Referring now to FIG. 4A, a reinforcing assembly 300 is introduced within the medical catheter 100, wherein the selective removal of portions of the polymeric jacket 210 and mechanical connection 202 of the reinforcing assembly 300 is performed at the attachment location 200. The reinforcing assembly 300, is a structural element within the medical catheter that facilitates steering control and maneuverability with the integration of reinforcing sleeves 302 which may act as a reinforced steering wire guidance system within the catheter shaft, allowing for controlled deflection of the distal portion 106 while providing support and stability to the proximal portion 104. The reinforcing assembly 300 is positioned within the proximal portion 104 of the medical catheter shaft 102 and extends into the distal portion 106 and comprises reinforcing sleeves 302 and a anchor ring 304, wherein the anchor ring 304 may be composed from a metallic material or of a similar alloy that comprises a circumferential groove 309 around the anchor ring. The reinforcing sleeves 302 may comprise a pair of diametrically opposed helical coils that extends longitudinally through the proximal portion 104 of the shaft. Further, the reinforcing sleeves 302 create dedicated passageways for one or more steering elements (e.g., wires, tendons, ribbons, and the like) 306, wherein the one or more steering element may comprise a first steering element 306a and a second steering element 306b. The one or more steering elements 306 are located in diametrically opposite positions and are operatively connected to the steering actuator 118 (FIG. 1) to facilitate the deflection of at least the deflection region 108 of the shaft 102 as known in the conventional manner. Additionally, the reinforcing sleeves 302 may provide radial and lateral support and guidance for the one or more steering elements 306 by encasing them within the helical coils. The protective structure may help minimize the impact of any compressive or any other forces that may occur during catheter manipulation allowing the steering elements to maintain their integrity and functionally for consistent and precise deflection of the deflection region 108.

Further, at a distal end of the reinforcing assembly 308, the reinforcing sleeves 302 are securely attached to the anchor ring 304, wherein the anchor ring 304 includes passageways for the one or more steering elements 306 extending from the proximal region to the distal region. The anchor ring 304 features a circumferential groove 309, wherein the circumferential groove may accommodate an attachment material such as an adhesive, polymeric material, or similar bonding materials. The anchor ring 304 may be positioned underneath and at center of, or in close proximity to the window 200 of the exposed reinforcing braid 205 wherein an attachment material is disposed through the window to fill the space of the circumferential groove 309 to establish a mechanical connection 202. The attachment material may flow through the exposed braid 205 into the groove 309, filling the voids or gaps and forming a strong bond that prevents slippage and maintains the integrity of the reinforcing assembly 300. The secure attachment allows for a seamless transfer of steering forces from the proximal portion 104 to the distal portion 106, allowing control and maneuverability of the medical catheter 100.

In some embodiments, the reinforcing assembly 300 may be adapted to suit specific catheter designs and applications. Variations in the number, configuration, and/or material composition of the reinforcing sleeves 302 and anchor ring 304 may be employed to optimize performance characteristics. For example, the helical coils of the reinforcing sleeves may be designed with different diameters and materials, or the anchor ring may be constructed of various materials to achieve the desired strength, and flexibility.

FIG. 4B introduces the articulation structure 400 that may be positioned within the distal portion 106 of the medical catheter shaft 102 and facilitates predictable, highly planar deflection of the deflection region 108 by resisting torsional forces on the shaft 102 that would otherwise tend to cause the deflection region 108 to deflect. The articulation structure 400 may comprise a circumferential groove 408 formed on its proximal end 404, wherein the circumferential groove 408 may accommodate an attachment material such as an adhesive, polymeric material, or similar bonding materials. The circumferential groove 408 of the articulation structure sits underneath and at center of, or in close proximity to the window 200 of the exposed groove braid 205 wherein an attachment material is disposed through the window 200 to fill the space of the circumferential groove 408 to establish a mechanical connection 202. The attachment material may flow through the exposed braid 205 into the groove 408, filling the voids or gaps and forming a strong bond that prevents slippage and maintains the integrity of the articulation structure 400.

In some embodiments, a separate ring component, similar to the anchor ring 304 used in the reinforcing assembly 300, can be attached to the proximal end 404 of the articulation structure wherein the ring has a circumferential groove that may be positioned underneath and at center, or in close proximity to the window 200 of the exposed braid 205. The ring of the articulation structure may securely be attached though the mechanical connection 202 by flowing an attachment material through the window 200 into the grooves of the ring in a similar manner as discussed for the anchor ring 304 in FIG. 4A.

Further, the articulation structure 400 may comprise series of interconnected plurality of longitudinally-arranged articulation elements that work together to achieve the desired deflection characteristics. These articulation elements may comprise a plurality of longitudinally arranged tubular segments, a plurality of connecting segments, plurality of articulation links, a plurality of joints, a plurality of reinforcing members, or combinations thereof. The articulation structure 400 may include one or more reinforcing members embedded within the articulation elements and extending through the articulation structure 400 wherein the reinforcing members may maintain a curved shape of the deflection region 108 during deflection and maintain repeatability of achieving the curved shape of the deflection region 108 during deflection. In an example embodiment the one or more reinforcing members may comprise a helically wound flat ribbon wire or stranded wire cables or of similar elements.

The articulation structure 400 may further comprise one or more steering wire lumens, wherein the steering wire lumens may be configured to receive one or more steering elements 306 that are operatively connected to the steering actuator 118 (FIG. 1) to facilitate the deflection of at least the deflection region 108 of the tubular shaft 102 as known in the conventional manner. The one or more steering wire lumens may also be circumferentially offset from the one or more reinforcing members by about 90 degrees. The specific design and arrangement of these elements can vary depending on the desired deflection profile and the intended application of the medical catheter 100.

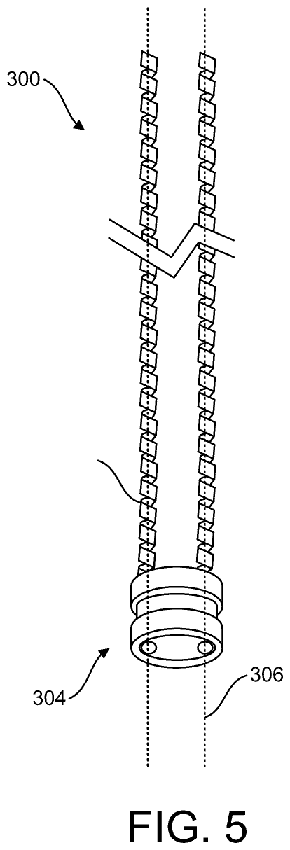

FIG. 5 is a top-view illustration of a reinforcing assembly 300 of the medical catheter 100, according to some embodiments. As shown, the reinforcing assembly 300 includes the reinforcing sleeves 302 wherein the reinforcing sleeves 302 create dedicated passageways for the one or more steering elements 306. The one or more steering elements 306 may be located in diametrically opposite positions and are operatively connected to the steering actuator 118 (FIG. 1) to facilitate the deflection of at least the deflection region 108 of the tubular shaft 102 as known in the conventional manner.

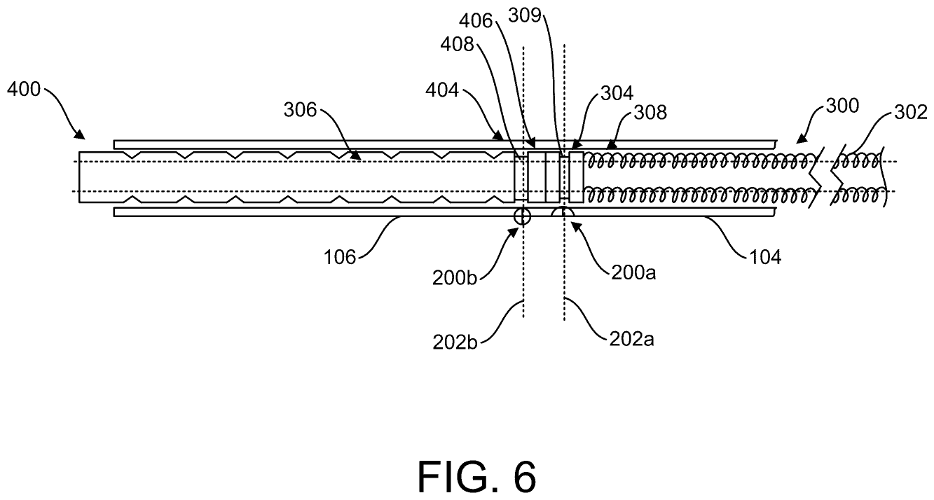

FIG. 6 introduces the creation of multiple windows of the exposed reinforcing braid 200a and 200b along the catheter shaft 102 to allow for mechanical connection 202a and 202b of more than one structural element. This approach allows for the integration of structural elements such as the reinforcing assembly 300 and the articulation structure 400 which may contribute to the overall steering performance and stability of the catheter 100. The process may involve selectively removing portions of the polymeric jacket 210 at specific locations along the catheter shaft 102 creating multiple windows of the exposed reinforcing braid 200a and 200b.

As shown in FIG. 6, the reinforcing assembly 300 is positioned such that the anchor ring 304 sits underneath and at center of, or in close proximity to the first window of exposed reinforcing braid 200a. The anchor ring 304 features a circumferential groove 309 that may accommodate an attachment material such as an adhesive, polymeric material, or similar bonding materials, wherein an attachment material is flowed through the window 200a to fill the space of the circumferential groove 309 and establish the first mechanical connection 202a. The attachment material may flow through the exposed braid 205 into the groove 309, filling the voids or gaps and forming a strong bond that prevents slippage and maintains the integrity of the reinforcing assembly 300.

Similarly, the articulation structure 400 may comprise a circumferential groove 408 formed on its proximal end 404, wherein the circumferential groove 408 may accommodate an attachment material such as an adhesive, polymeric material, or similar bonding materials. The circumferential groove 408 of the articulation structure sits underneath and at center of, or in close proximity to the second window 200b of the exposed groove braid 205 wherein an attachment material is flowed through the window 200b to establish the second mechanical connection 202b. The attachment material is flowed through the window 200b to fill the space of the circumferential groove 408. The attachment material may flow through the exposed braid 205 into the groove 408, filling the voids or gaps and forming a strong bond that prevents slippage and maintains the integrity of the articulation structure 400. In some embodiments, a separate ring component, similar to the anchor ring 304 used in the reinforcing assembly 300, can be attached to the proximal end 404 of the articulation structure wherein the ring has a circumferential groove that may be positioned underneath and at center, or in close proximity to the window 200b of the exposed braid 205. The ring may be securely attached though the mechanical connection 202b by flowing an attachment material through the window 200b into the grooves of the ring.

The mechanical connections 202a and 202b may be done by flowing an attachment material through the windows 200a and 200b of the exposed braids 205 into the circumferential grooves of the reinforcing assembly and articulation structure, wherein the attachment material may be an adhesive, polymeric material, or similar bonding materials that may enhance the connection of the articulation structure and the structural elements to the polymeric jacket. As the attachment material fills the grooves 309 of the reinforcing assembly and 408 of the articulation structure, it creates a direct contact of the between these structural elements and to the polymeric jacket 210. This direct contact allows the attachment material to adhere to both the structural elements (the reinforcing assembly and the articulation structure) and the polymeric jacket 210, effectively locking them in place and creating a strong bond with the polymeric jacket 210. The attachment material, upon curing or solidifying, may become an integral part of the catheter shaft 102, seamlessly integrating the reinforcing assembly 300 and the articulation structure 400 into the shaft 102.

The positioning of the windows 200a and 200b of the exposed reinforcing braid, along with the placement of the reinforcing assembly 300 and articulation structure 400 can be tailored based on the specific design requirements of the catheter 100. Factors such as the desired deflection profile, the length of the distal portion 106, and the overall catheter stiffness can influence the optimal placement of these components. In some embodiments, additional windows 200 of the exposed braid may be created to accommodate mechanical connection to other structural elements or to provide multiple points for mechanical connection to a single structural element. As such, a user may strategically remove the polymeric material by laser ablation or of similar removal process such that length, width, and depth of the window 200 of the exposed reinforcing braid 205 may be tailored to a desired size to accommodate the integration of different structural elements to the shaft. Moreover, the pattern and arrangement of the window 200 of the exposed reinforcing braids 205 can be modified to achieve specific performance characteristics. For example, helical, staggered or other arrangements of the window 200 of the exposed reinforcing braids 205 could be utilized to optimize the flexibility and torsional rigidity of the catheter shaft 102 in specific region to allow for the secure integration of structural elements through the window 200 of the reinforcing braid 205 while maintaining a smooth, continuous profile along the catheter shaft 102 contributing to the overall performance, maneuverability, and stability of the steerable medical catheter 100.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical catheter comprising:
a handle configured for manipulation by a user; and
a flexible shaft comprising a reinforcing braid and a polymeric jacket disposed over the reinforcing braid, the flexible shaft having a proximal portion and a distal portion, the shaft further comprising a reinforcing assembly extending within the proximal portion of the shaft, wherein the reinforcing assembly comprises an anchor ring and a pair of diametrically opposed reinforcing sleeves attached to and extending proximally from the anchor ring within the proximal portion of the shaft, and wherein the anchor ring includes a circumferential groove,
wherein the anchor ring is mechanically connected to the polymeric jacket and the reinforcing braid via a first mechanical connection establishing a secure bond between the polymeric jacket, the reinforcing braid and the structural element, and
wherein the first mechanical connection is formed by disposing an attachment material into the circumferential groove in the anchor ring through a first window formed in the polymeric jacket.

2. The medical catheter of claim 1, wherein the attachment material is an adhesive, a polymeric material, or other similar bonding materials.

3. The medical catheter of claim 2, further comprising a pair of diametrically opposed steering elements each extending through a respective one of the reinforcing sleeves, wherein the reinforcing sleeves each provide radial and lateral support for the steering elements extending therethrough.

4. The medical catheter of claim 3, wherein the anchor ring includes a pair of diametrically opposed passageways aligned with the passageways of the reinforcing sleeves, and when each of the steering elements extends through a respective one of the passageways in the anchor ring.

5. The medical catheter of claim 4, wherein the shaft further comprises an articulation structure positioned within the distal portion of the shaft, the articulation structure having a proximal end positioned distally adjacent to the anchor ring.

6. The medical catheter of claim 5, wherein the proximal end of the articulation structure includes a circumferential groove and is mechanically connected to the polymeric jacket and the reinforcing braid via a second mechanical connection formed by disposing an attachment material into the circumferential groove in the proximal end of the articulation structure through a second window formed in the polymeric jacket.

7. The medical catheter of claim 6, wherein the articulation structure includes a plurality of longitudinally-arranged articulation elements, and a pair of diametrically opposed steering wire lumens, each of the steering wire lumens being aligned with a respective one of the reinforcing sleeves, and wherein each of the steering elements extends through a respective one of the steering wire lumens.

8. A medical catheter comprising:
a handle configured for manipulation by a user; and
a flexible shaft comprising a reinforcing braid and a polymeric jacket disposed over the reinforcing braid, the flexible shaft having a proximal portion and a distal portion, the shaft further comprising a reinforcing assembly extending within the proximal portion of the shaft, wherein the reinforcing assembly comprises an anchor ring and a pair of diametrically opposed reinforcing sleeves attached to and extending proximally from the anchor ring within the proximal portion of the shaft, and wherein the anchor ring includes a circumferential groove,
wherein an adhesive attachment material is disposed within the circumferential groove in the anchor ring and mechanically connects the anchor ring to the reinforcing braid and the polymeric jacket.

9. The medical catheter of claim 8, further comprising a pair of diametrically opposed steering elements each extending through a respective one of the reinforcing sleeves, wherein the reinforcing sleeves each provide radial and lateral support for the steering elements extending therethrough.

10. The medical catheter of claim 9, wherein the anchor ring includes a pair of diametrically opposed passageways aligned with the passageways of the reinforcing sleeves, and when each of the steering elements extends through a respective one of the passageways in the anchor ring.

11. The medical catheter of claim 10, wherein the reinforcing sleeves comprise helical coils.

12. The medical catheter of claim 10, wherein the shaft further comprises an articulation structure positioned within the distal portion of the shaft, the articulation structure having a proximal end positioned distally adjacent to the anchor ring.

13. The medical catheter of claim 12, wherein the proximal end of the articulation structure includes a circumferential groove and is mechanically connected to the polymeric jacket and the reinforcing braid via an adhesive attachment material disposed within the circumferential groove in the proximal end of the articulation structure.

14. The medical catheter of claim 13, wherein the articulation structure includes a plurality of longitudinally-arranged articulation elements, and a pair of diametrically opposed steering wire lumens, each of the steering wire lumens being aligned with a respective one of the reinforcing sleeves, and wherein each of the steering elements extends through a respective one of the steering wire lumens.

15. The medical catheter of claim 14, wherein the articulation structure further includes a pair of diametrically opposed reinforcing members extending along a length of the articulation structure, wherein each of the reinforcing members is located 90 degrees from the steering wire lumens of the articulation structure.

16. A method of producing a medical catheter comprising:
providing a flexible shaft comprising a reinforcing braid and a polymeric jacket disposed over the reinforcing braid, the flexible shaft having a proximal portion, and a distal portion;
removing a portion of the polymeric jacket to create a window of the exposed reinforcing braid;
positioning a structural element within the shaft adjacent to the window;
connecting the structural element to the polymeric jacket by disposing an attachment material through the window of the exposed reinforcing braid to establish a secure bond between the structural element, the reinforcing braid and the polymeric jacket; and
securing a handle to the proximal portion of the catheter shaft, the handle configured for manipulation by a user.

17. The method of claim 16, wherein the structural element comprises a reinforcing assembly comprising an anchor ring and a pair of diametrically opposed reinforcing sleeves attached to and extending proximally from the anchor ring within the proximal portion of the shaft, and wherein:
positioning the structural element includes positioning the anchor ring adjacent to the window; and
connecting the structural element to the polymeric jacket comprises disposing an adhesive material through the window to establish a secure bond between the anchor ring, the reinforcing braid and the polymeric jacket.

18. The method of claim 17, wherein the anchor ring includes a circumferential groove, and wherein connecting the structural element to the polymeric jacket includes disposing the adhesive material through the window into the circumferential groove.

19. The method of claim 18, wherein the reinforcing sleeves comprise helical coils and are each configured to provide radial and lateral support for a steering element extending therethrough.

20. The method of claim 16, wherein the structural element is an articulation structure having a proximal end having a circumferential groove formed thereon, a plurality of longitudinally-arranged articulation elements, and one or more steering wire lumens extending through the articulation structure, and wherein:
positioning the structural element includes positioning the proximal end of the articulation structure adjacent to the window; and
connecting the structural element to the polymeric jacket comprises disposing an adhesive material through the window and into the circumferential groove to establish a secure bond between the proximal end of the articulation structure, the reinforcing braid and the polymeric jacket.
